(19) United States
(12) Patent Application Publication
(10) Pub. No.: US 2025/0367264 A1
(43) Pub. Date: Dec. 4, 2025

(54) GLP-1 ANALOGUE COMPOSITIONS AND PREPARATION METHOD

(71) Applicant: Galenicum Health S.L.U., Esplugues de Llobregat (ES)

(72) Inventors: Luis Gómez COELLO, San Agustín de Guadalix (ES); Jamie Osuna LÓPEZ, Madrid (ES)

(21) Appl. No.: 19/019,721

(22) Filed: Jan. 14, 2025

(30) Foreign Application Priority Data
May 31, 2024 (EP) 24382587

(51) Int. Cl. A61K 38/26; C07K 14/605

(52) U.S. Cl. CPC A61K 38/26; C07K 14/605

(57) ABSTRACT

Disclosed herein is a method for the preparation of a pharmaceutical composition comprising a GLP-1 agonist, a tonicity modifier, a buffering agent and a preservative which comprises the following steps: preparing a final solution comprising the tonicity modifier, the buffering agent, the preservative, the GLP-1 agonist and water for injection; and adjusting the pH of the final solution between 7.5-8.5 with a pH adjuster; wherein the final solution is prepared at a temperature between 2-15° C.

### RELATED APPLICATION

This application claims priority to European patent application EP24382587, filed on May 31, 2024.

### FIELD

Disclosed herein is a method for the preparation of a pharmaceutical composition comprising a GLP-1 agonist, as well as a pharmaceutical composition prepared according to the method and to a cartridge comprising the pharmaceutical composition.

### BACKGROUND

The development of pharmaceutical compositions comprising therapeutic peptides such as glucagon-like peptide-1 (GLP-1) analogues or agonists is of significant interest due to their potential in treating conditions like diabetes and obesity. GLP-1 analogues are susceptible to degradation when formulated as pharmaceuticals, and the process of preparing these compositions requires careful consideration of factors such as stability, efficacy, and safety of the product. However, these peptides are prone to chemical instability, such as oxidation or hydrolysis, which may affect their structural integrity and therapeutic function, and also to physical instability, such as aggregation or precipitation, which may lead to reduced bioavailability and possible immunogenic reactions. The combination of these factors makes the development of stable GLP-1 analogue formulations a complex and critical task in pharmaceutical sciences.

Particularly, fibril formation, a process by which a GLP-1 analogue tends to form well-ordered, thread-like macromolecular aggregates, poses a significant challenge in a GLP-1 analogue formulation. In the literature, heat treatment of GLP-1 analogue solutions has been suggested for increasing the shelf life and stability of the pharmaceutical solutions due to fibril formation.

Fibrils in parenteral compositions present several significant disadvantages that impact the safety, efficacy, and regulatory compliance of pharmaceutical products. Immunogenicity is a major concern, as fibrils can provoke an immune response, potentially leading to adverse reactions in patients, ranging from mild allergic responses to severe immunological complications. The presence of fibrils also signifies a loss of therapeutic efficacy, as aggregated proteins are often denatured and incapable of performing their intended biological functions. This aggregation indicates instability, compromising the product's shelf-life and necessitating stringent storage conditions. Additionally, the physical presence of fibrils can pose safety risks, such as causing blockages in blood vessels or tissues, which could lead to serious health issues like embolism or inflammation. Hence, minimizing fibril formation is crucial for ensuring the overall integrity and success of parenteral pharmaceutical applications.

According to WO2006051110A2, fibrillation in GLP-1 analogue solutions can be reduced by heating the solution of said GLP-1 analogues between 50° C. and 95° C., at a pH between 8.0 to 10.5 and then continue the heating for between 3 minutes and 180 minutes. This method would allow the fibrils to dissolve in their initial state and delay its formation.

WO2020127476A1 discloses a method for the preparation of pharmaceutical solution comprising a GLP-1 analogue which involves heating the solution to a temperature of 26-49° C.

However, due to the importance of therapeutic peptides, there is a continuous need for innovative approaches that can enhance the stability of GLP-1 analogue formulations, thereby improving their therapeutic effectiveness and enhancing their stability in the pharmaceutical product lifecycle.

The disclosed method is directed towards addressing the shortcomings and challenges outlined above.

### SUMMARY

Disclosed herein is a method for the preparation of a pharmaceutical composition comprising a GLP-1 agonist having an increased stability and less tendency to form fibrils.

Heat treatment of peptide solutions has been described in the literature for providing stable pharmaceutical solutions. However, the inventors have surprisingly found that stable pharmaceutical formulations comprising a GLP-1 agonist can be prepared without involving thermal treatment of the solutions. It has been observed that GLP-1 agonist compositions prepared according to a method disclosed herein have a reduced number of fibrils and impurity content, avoiding fibril and/or impurity formation in the manufacturing process.

In addition, the method disclosed herein provides safe and stable pharmaceutical compositions that are cost-effective to manufacture at industrial scale.

Each of the aspects and embodiments of each of the methods, compositions, and cartridges disclosed herein may be combined with one or more aspects and embodiments of each of the methods, compositions, and cartridges.

A first aspect concerns a method for the preparation of a pharmaceutical composition comprising a GLP-1 agonist, a tonicity modifier, a buffering agent, and a preservative which comprises the following steps:
a. preparing a final solution comprising the tonicity modifier, the buffering agent, the preservative, the GLP-1 agonist and water for injection; and
b. adjusting the pH of the final solution between 6.5-8.5, with a pH adjuster;
   wherein the final solution is prepared at a temperature between 2-15° C.

The pharmaceutical solutions prepared according to the method of the first aspect have lower number of fibrils, i.e., less fibrillation over time and less individual and total impurities than pharmaceutical solutions which had undergone heat treatment.

A second aspect concerns a pharmaceutical composition obtainable by the method according to the first aspect.

A third aspect concerns a cartridge comprising the pharmaceutical composition according to the second aspect.

### DETAILED DESCRIPTION

As used in this disclosure, the following words, phrases, and symbols are generally intended to have the meanings as set forth below, except to the extent that the context in which they are used indicates otherwise.

All percentages are expressed by weight (w/w) and as used herein are referred to the total weight of the composition, unless specifically noted otherwise.

The term “active ingredient” as used herein refers to a therapeutically active peptide, as well as any pharmaceutically acceptable salts, hydrates, and solvates of the compound.

The term “buffer” as used herein refers to a chemical compound in a pharmaceutical composition that reduces the tendency of pH of the composition to change over time as would otherwise occur due to chemical reactions. Examples of buffers include sodium dihydrogen phosphate, dibasic sodium phosphate, sodium phosphate, phosphoric acid, acetic acid, sodium acetate, sodium carbonate, sodium bicarbonate, carbonic acid, citrate, citric acid, meglumine, glycine, histidine, lysine, arginine, asparagine, glutamic acid, sodium glutamate, tris (hydroxymethyl)-aminomethane, methionine, Hepes, maleic acid, malic acid, lactate, etc.

The term “pH adjuster” as used herein refers to pharmaceutically acceptable excipients which are added to the solution of the active ingredient to adjust the pH to a certain value. Such pH adjusters can be alkaline or acid agents and may comprise inorganic salts as well as organic acids or salts of organic acids. Examples of preferred pH adjusters are HCl and/or NaOH.

The term “pharmaceutically acceptable” as used herein indicates that the substance or composition must be compatible chemically and/or toxicologically, with the other ingredients in the composition, and/or the mammal (e.g., human) being treated therewith.

The term “pharmaceutical composition” as used herein means a product comprising an active ingredient or a salt thereof together with pharmaceutical excipients such as buffer, preservative and tonicity modifier, said pharmaceutical composition being useful for treating, preventing or reducing the severity of a disease or disorder by administration of said pharmaceutical composition to a person (e.g., human). The expression “pharmaceutical composition” may also be referred to as a “pharmaceutical formulation.”

The term “preservative” as used herein refers to a chemical compound which is added to a pharmaceutical composition to prevent or delay microbial activity (growth and metabolism). Examples of pharmaceutically acceptable preservatives include, but are not limited to phenol, m-cresol, methyl p-hydroxybenzoate, propyl p-hydroxybenzoate), benzoic acid, benzyl alcohol, benzyl benzoate, 2-phenoxyethanol, butyl p-hydroxybenzoate, 2-phenylethanol, benzyl alcohol, chlorobutanol, acetone sodium bisulfite, benzalkonium chloride, benzethonium chloride, and thiomerosal.

The term “stable” as used herein refers to any pharmaceutical composition comprising the active ingredient having a sufficient physical and chemical stability to allow storage under any of the general storage conditions as defined by ICH Q1A (R2).

The term “tonicity modifier” as used refers to a chemical compound in a pharmaceutical composition that serves to modify the osmotic pressure of the pharmaceutical composition so that the osmotic pressure becomes closer to that of human plasma. The tonicity modifier is also known in the art as “isotonicity agent”. Isotonicity agents include but are not limited to mannitol, sorbitol, lactose, propylene glycol, dextrose, trehalose, sodium chloride, potassium chloride, glycerol, glycerin, etc.

The quantitative determination of liraglutide by HPLC analysis was carried out using ACQUITY UPLC H-Class PLUS system, Aeris Peptide XB C18 100 Å 4.6 mm×250 mm, 3.6 μm column was used. A Security Guard ULTRA cartridge, UHPLC C18-Peptide 4.6 mm ID pre-column was used. The apparatus was equipped with a manual injector and UV detector. The injection valve was a Rheodyne with a capacity of 20 μL. Mobile phase A (0.5% TFA in Water:Methanol (95:5, v/v %)) and Mobile phase B (0.5% TFA in Acetonitrile:Methanol:Water (90:5:5, v/v/v %) with 6.0 mL NH3 30%) were used at a ratio 40:60. As a diluent, 0.025% v/v Ammonia in water was used. The mobile phases were filtered through a 0.45 μm membrane filter and sonicated before use. It was pumped through the column at a flow rate of 0.8 mL/min. Injection volume was 10 μL and the column was maintained at 25° C. The detection was monitored at 220 nm and the run time was set as 35 minutes. The amount of liraglutide in the samples was determined by comparison with appropriate external standard curves obtained applying the least square linear regression analysis.

The quantitative determination of liraglutide impurities by UPLC was carried out using an ACQUITY UPLC H-Class PLUS system, Acquity UPLC Peptide CSH C18 130 Å 2.1×150 mm, 1.7 μm (Two columns connected in series with column coupler) column was used. An Acquity UPLC Peptide CSH C18 Vanguard Pre-Column 130 Å 5×2.1 mm, 1.7 μm pre-column was used. Mobile phase A (Buffer solution 4 mM:Methanol:TFA (950:50:1, v/v/v %) adjusted to pH 7.00 with Ammonia solution) and Mobile phase B (Acetonitrile:Methanol:Water:TFA (500:450:50:1, v/v/v/V %) were used at a ratio 26:74. As a diluent, 0.025% v/v Ammonia in water was used. The mobile phases were filtered through a 0.45 μm membrane filter and sonicated before use. It was pumped through the column at a flow rate of 0.07 mL/min. Injection volume was 4 μL and the column was maintained at 25° C. The detection was monitored at 215 nm and the run time was set as 145 minutes. The percentage of other impurities are calculated by the following equation:

$$\% Impurity = ( As \times Wst \times P \times fds \times Ds \times Cf ) / ( Ast \times Ws \times fdst \times C )$$

Where,
As: Impurity peak area in test solution
Ast: Liraglutide peak area in reference solution
Wst: Weight of Liraglutide standard in Reference Solution (mg)
Ws: Weight of sample in test solution (g)
P: Purity of Liraglutide standard as is (%), into account KF determination before use (H)

$$P = P Liraglutide \times ( 100 - ( H ) ) / 100$$

fdst: Dilution factor of Reference Solution (2500)
fdS: Dilution factor of test solution (10)
Ds: Density of product (g/mL)
C: Content of Liraglutide in 1 mL, in mg (6.0)
Cf: Correction factor (1/RRF=Liraglutide RF/Impurity RF)

The term “unknown impurity” as used herein refers to an impurity of unknown structure having a specific relative retention time (RRT or t_{Rr}) in each case. The percentage of each impurity is calculated as explained above from the results of the analysis under the UPLC conditions set forth above.

The term “analogue” as used herein referring to a peptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide.

The term “derivative” as used herein in relation to a parent peptide means a chemically modified parent protein or an analogue thereof, wherein at least one substituent is not present in the parent protein or an analogue thereof, i.e. a parent protein which has been covalently modified. Typical modifications are amides, carbohydrates, alkyl groups, acyl groups, esters, PEGylations and the like.

The term “GLP-1 agonist” or “GLP-1 analogues”, as used herein refer to a class of active ingredients that reduce blood sugar and energy intake by activating the GLP-1 receptor. They mimic the actions of the endogenous incretin hormone GLP-1 that is released by the gut after eating. Examples of GLP-1 agonists include dulaglutide, albiglutide, liraglutide, semaglutide, exenatide, lixisenatide, and tirzepatide. It will be appreciated that a GLP-1 agonist may include one or more amino acids that have an ionizable moiety associated with a counterion, and thus, reference of a particular GLP-1 agonist is a reference to a pharmaceutically acceptable salt thereof.

All percentages, parts and ratios herein used are by weight unless specifically noted otherwise. As used herein, the term “about” refers to a range that is ±10% (alternatively, ±5%, or ±1%) of a value with which the term is associated. A numerical value not associated with the term “about” should not be taken to mean that there is no associated variation associated with the numerical value.

Unless otherwise indicated, all the analytical methods are carried out according to the European Pharmacopoeia 10th edition.

The inventors have determined that a pharmaceutical composition prepared according to a method disclosed herein has a lower level of impurities and lower quantity of fibrils over time than when prepared by other methods involving heat treatment. The heat treatment of the fibrils is in principle carried out with the aim of dissolving the fibrils formed and avoiding its appearance. Surprisingly, the tendency to form fibrils is reduced in a method disclosed herein, which employs low temperatures in the preparation of the pharmaceutical compositions.

The first aspect relates to a method for the preparation of a pharmaceutical composition comprising a GLP-1 agonist, a tonicity modifier, a buffering agent, and a preservative which comprises the following steps:
a. preparing a final solution comprising the tonicity modifier, the buffering agent, the preservative, the GLP-1 agonist and water for injection; and
b. adjusting the pH of the final solution between 6.5-8.5 (or 7.5-8.5) with a pH adjuster;
   wherein the final solution is prepared at a temperature between 2-15° C. (or any temperature in between, such as 2-10° C., 2-8° C., or 3-6° C.

In one embodiment, the final solution may be prepared by mixing a first solution comprising the tonicity modifier, the buffering agent, the preservative, and water for injection; and a second solution comprising a GLP-1 agonist and water for injection, wherein the first and second solutions are prepared at a temperature between 2-15° C. (or any temperature in between, such as 2-10° C., 2-8° C., or 3-6° C.

In another embodiment, only one final solution is prepared by mixing the tonicity modifier, the buffering agent, the preservative, the GLP-1 agonist, and water for injection at a temperature between 2-15° C. and any temperature in between, such as between 2-10° C., 2-8° C., or 3-6° C.

In another embodiment, the method may further comprise bubbling nitrogen gas in any of the first, second and/or final solutions, such that the dissolved oxygen content is <2.0 ppm. It has been determined that bubbling nitrogen gas helps to avoid the formation of fibrils, particularly when the dissolved oxygen content is kept below 2.0 ppm.

In another embodiment, the method may further comprise filtering the final solution through a 0.2 μm (e.g., 0.22 μm) pore size filter. Filtering the final solution through a 0.2 μm pore size filter delays the formation of fibrils.

In an embodiment, the GLP-1 agonist may comprise dulaglutide, albiglutide, liraglutide, semaglutide, exenatide, lixisenatide, tirzepatide or mixtures thereof (alternatively the GLP-agonist may comprise liraglutide, semaglutide, or tirzepatide or the GLP-1 agonist comprises liraglutide.

In an embodiment, the tonicity modifier comprises mannitol, sorbitol, lactose, propylene glycol dextrose, trehalose, sodium chloride, potassium chloride, glycerol, glycerin, or mixtures thereof. In yet another embodiment, the tonicity modifier comprises propylene glycol.

In an embodiment, the buffering agent comprises sodium dihydrogen phosphate, dibasic sodium phosphate, sodium phosphate, phosphoric acid, acetic acid, sodium acetate, sodium carbonate, sodium bicarbonate, carbonic acid, citrate, citric acid, meglumine, glycine, histidine, lysine, arginine, asparagine, glutamic acid, sodium glutamate, tris (hydroxymethyl)-aminomethane, methionine, Hepes, maleic acid, malic acid, lactate, or mixtures thereof. In one embodiment, the buffering agent comprises dibasic sodium phosphate.

In an embodiment, the preservative comprises phenol, m-cresol, methyl p-hydroxybenzoate, propyl p-hydroxybenzoate), benzoic acid, benzyl alcohol, benzyl benzoate, 2-phenoxyethanol, butyl p-hydroxybenzoate, 2-phenylethanol, benzyl alcohol, chlorobutanol, acetone sodium bisulfite, benzalkonium chloride, benzethonium chloride and thiomerosal, or any combinations thereof. In another embodiment, the preservative comprises phenol.

In an embodiment, the method further comprises filling aseptically the final solution into cartridges flushed with nitrogen gas. The nitrogen gas flushed in the cartridges delays the appearance of fibrils. In an embodiment, no gas bubbles remain inside the cartridges. The inventors have surprisingly found that when gas bubbles remain in the cartridge, even if nitrogen is flushed, the concentration of fibrils is higher in the pharmaceutical composition disclosed herein.

A second aspect relates to a pharmaceutical composition obtainable by the method according to the first aspect.

In an embodiment according to the second aspect, the pharmaceutical composition has an oxygen content below 2.0 ppm.

A third aspect relates to a cartridge comprising the pharmaceutical composition according to the second aspect.

Further embodiments can be found in the following numbered clauses:

Clause 1. A method for the preparation of a pharmaceutical composition comprising a GLP-1 agonist, a tonicity modifier, a buffering agent and a preservative which comprises the following steps:
a. preparing a final solution comprising the tonicity modifier, the buffering agent, the preservative, the GLP-1 agonist and water for injection; and
b. adjusting the pH of the final solution between 6.5-8.5 (or between 7.5-8.5), with a pH adjuster;
   wherein the final solution is prepared at a temperature between 2-15° C. or any temperature in between, such as, 2-10° C., 2-8° C., or 3-6° C.

Clause 2. The method of clause 1, wherein the final solution is prepared by mixing a first solution comprising the tonicity modifier, the buffering agent, the preservative, and water for injection; and a second solution comprising a GLP-1 agonist and water for injection, wherein the first and second solutions are prepared at a temperature between 2-15° C. or any temperature in between, such as 2-10° C., 2-8° C., or 3-6° C.

Clause 3. The method of any of the preceding clauses, comprising bubbling nitrogen gas in any of the first, second and/or final solution, where the dissolved oxygen content may be <2.0 ppm.

Clause 4. The method of any of the preceding clauses, comprising filtering the final solution through a 0.2 μm pore size filter.

Clause 5. The method of any of the preceding clauses, wherein the GLP-1 agonist comprises dulaglutide, albiglutide, liraglutide, semaglutide, exenatide, lixisenatide, tirzepatide, or mixtures thereof; alternatively, the GLP-1 agonist comprises liraglutide, semaglutide, or tirzepatide; or the GLP-1 agonist comprises liraglutide.

Clause 6. The method of any of the preceding clauses, wherein the tonicity modifier comprises mannitol, sorbitol, lactose, propylene glycol dextrose, trehalose, sodium chloride, potassium chloride, glycerol, glycerin or mixtures thereof; alternatively, the tonicity modifier comprises propylene glycol.

Clause 7. The method of any of the preceding clauses, wherein the buffering agent comprises sodium dihydrogen phosphate, dibasic sodium phosphate, sodium phosphate, phosphoric acid, acetic acid, sodium acetate, sodium bicarbonate, carbonic acid, sodium carbonate, citrate, citric acid, meglumine, glycine, histidine, lysine, arginine, asparagine, glutamic acid, sodium glutamate, tris (hydroxymethyl)-aminomethane, methionine, Hepes, maleic acid, malic acid, lactate or mixtures thereof; alternatively, the buffering agent comprises dibasic sodium phosphate.

Clause 8. The method of any of the preceding clauses, wherein the preservative comprises phenol, m-cresol, methyl p-hydroxybenzoate, propyl p-hydroxybenzoate, benzoic acid, benzyl alcohol, benzyl benzoate, 2-phenoxyethanol, butyl p-hydroxybenzoate, 2-phenylethanol, benzyl alcohol, chlorobutanol, acetone sodium bisulfite, benzalkonium chloride, benzethonium chloride and thiomerosal, or any combinations thereof; alternatively, the preservative comprises phenol.

Clause 9. The method of any of the preceding clauses, further comprising filling aseptically the final solution into cartridges flushed with nitrogen gas.

Clause 10. The method of any of the preceding clauses, wherein no gas bubbles remain inside the cartridges.

Clause 11. A pharmaceutical composition obtainable by the method of any one of clauses 1-10.

Clause 12. A pharmaceutical composition of clause 11, wherein the fibrils concentration in the pharmaceutical composition after 1-month storage at 30° C. and 65% relative humidity is less than 0.02 μM as measured by Thioflavin T (ThT) Fibrillation Estimation Assay, wherein the final solution is prepared at a temperature between 2-15° C. (alternatively at a temperature between 2-10° C., 2-8° C., or 3-6° C.), wherein the temperature during the preparation of the composition does not exceed 15° C. (or does not exceed 10° C., or 8° C.).

Clause 13. A pharmaceutical composition of any one of clauses 11-12, wherein the oxygen content is <2.0 ppm.

Clause 14. A cartridge comprising a pharmaceutical composition of any one of clauses 11-13.

Clause 15. A pharmaceutical composition comprising a GLP-1 agonist, a tonicity modifier, a buffering agent, and a preservative; wherein the oxygen content is <2.0 ppm.

Clause 16. The pharmaceutical composition of clause 15, wherein the fibrils concentration after 1-month storage at 30° C. and 65% relative humidity is less than 0.02 PM as measured by Thioflavin T (ThT) Fibrillation Estimation Assay, wherein the final solution is prepared at a temperature between 2-15° C. (or between 2-10° C., 2-8° C., or 3-6° C.), wherein the temperature during the preparation of the composition does not exceed 15° C. (or does not exceed 10° C. or 8° C.).

Clause 17. A method of any of the clauses 1-10, wherein the final solution temperature does not exceed 15° C.

Clause 18. A method of any of the clauses 1-10, wherein the final solution temperature does not exceed 14° C.

Clause 19. A method of any of the clauses 1-10, wherein the final solution temperature does not exceed 13° C.

Clause 20. A method of any of the clauses 1-10, wherein the final solution temperature does not exceed 12° C.

Clause 21. A method according to any of the clauses 1-10, wherein the final solution temperature does not exceed 11° C.

Clause 22. A method of any of the clauses 1-10, wherein the final solution temperature does not exceed 10° C.

Clause 23. A method of any of the clauses 1-10, wherein the final solution temperature does not exceed 9° C.

Clause 24. A method of any of the clauses 1-10, wherein the final solution temperature does not exceed 8° C.

Clause 25. A pharmaceutical composition comprising a GLP-1 agonist, a tonicity modifier, a buffering agent, and a preservative; wherein the fibrils concentration in the pharmaceutical composition after 1-month storage at 30° C. and 65% relative humidity is less than 0.02 μM as measured by Thioflavin T (ThT) Fibrillation Estimation Assay.

Clause 26. The pharmaceutical composition of clause 25, wherein the pH of said composition is between 6.5-8.5 (or between 7.5-8.5).

Clause 27. The pharmaceutical composition of any of clauses 25-26, wherein the oxygen content is <2.0 ppm.

Clause 28. The pharmaceutical composition of any of clauses 25-27, wherein said composition is manufactured by preparing a final solution at a temperature between 2-15° C. (or between 2-10° C., 2-8° C., or 3-6° C.), wherein the temperature during the preparation of the composition does not exceed 15° C. (or does not exceed 10° C. or 8° C.).

Clause 29. A cartridge comprising a pharmaceutical composition of any of the clauses 25 to 28.

One will appreciate that the expression “comprising” may be replaced with the expression “consisting of” without departing from the meaning of the embodiment or aspect. Further, one will appreciate that listed ingredients may be alternatively expressed using Markush group language, using the expression “selected from the group consisting of . . . .”

### EXAMPLES

### Example 1. Preparation of Pharmaceutical Compositions Comprising Liraglutide

Two pharmaceutical compositions comprising Liraglutide where prepared with the following concentrations of GLP-1 agonist, tonicity modifier, buffering agent, preservative and water for injection (WFI):

| | | | Dibasic | | |
|---|---|---|---|---|---|
| | Liraglu- | Propylene | sodium | | |
| Composition | tide | glycol | phosphate | Phenol | WFI |
| GLN-1 | 0.60 | 1.40 | 0.14 | 0.55 | to 100.00 |
| Comp. example 1 | 0.60 | 1.40 | 0.14 | 0.55 | to 100.00 |

The amount of the different components is expressed as percentages by weight (w/w %) of the total composition.

Both formulations were prepared through different methods. Formulation “GLN-1” was prepared according to the method described herein. The formulation “Comp. example 1” was prepared as disclosed in WO2006051110A2 except that nitrogen gas was bubbled through solutions.

Formulation “GLN-1” was manufactured by preparing a first solution comprising propylene glycol, dibasic sodium phosphate, phenol and water for injection. In another container, a second solution comprising liraglutide and water for injection was prepared. Then, a final solution was prepared by mixing the first and the second solution. The first, second and final solutions were prepared at a temperature between 2-8° C. Nitrogen gas was bubbled in the first, second and final solution, to maintain the dissolved oxygen content below 2.0 ppm. Then, the pH of the final solution was adjusted to 8.15 with HCl and/or NaOH. Eventually, the final solution was filtered through a 0.22 μm filter.

Formulation “Comp. example 1” was manufactured by preparing a first solution comprising propylene glycol, dibasic sodium phosphate, phenol and WFI. In another container, a second solution comprising liraglutide and WFI was prepared and heated to 60-70° C. and kept for 30 minutes at this temperature. After heat treatment, the second solution was cooled to 22° C. Afterwards, the first and second solutions were mixed to prepare the final solution, and pH adjusted to 8.15 using HCl and/or NaOH. Nitrogen gas was bubbled in the first, second and final solution, to maintain the dissolved oxygen content below 2.0 ppm. Finally, the formulation was filtered through a 0.22 μm filter.

### Example 2. Impurity Content

Both formulations obtained in Example 1 were analysed by UPLC as described above for impurity content at two different times, freshly prepared (t=0) and at 1 month stored at 25° C./60% RH. The following results were obtained:

| | |
|---|---|
| GLN | Comp. Ex. 1 |

| Impurities (RRT) | t = 0 | t = 1 | t = 0 | t = 1 |
|---|---|---|---|---|
| Unknown impurity (1.08) | 0.16 | 0.14 | ND | ND |
| Unknown impurity (1.106) | ND | ND | 0.16 | ND |
| Imp E (1.11) | 0.10 | 0.18 | 0.11 | 0.540 |
| Unknown impurity (1.14) | 0.18 | 0.04 | 0.14 | 0.750 |
| Imp D (1.17) | ND | 0.07 | ND | 0.060 |
| Unknown impurity (1.20) | 0.16 | 0.06 | 0.11 | 0.520 |
| Unknown impurity (1.22) | 0.05 | ND | 0.05 | 0.620 |
| Unknown impurity (1.23) | ND | ND | ND | 0.460 |
| Unknown impurity (1.36) | ND | 0.09 | ND | 2.950 |
| Imp F (1.41) | ND | 0.16 | ND | 0.370 |
| Imp G (1.45) | ND | 0.16 | ND | 0.350 |
| Imp I (1.50) | ND | ND | 0.04 | 0.030 |
| Imp H (1.52) | 0.11 | 0.14 | 0.35 | 0.500 |
| Total impurities (%) | 0.76 | 1.04 | 0.96 | 7.15 |

All amounts of impurities are expressed as a percentage, “ND” corresponds to not detected. As it can be seen, the amount of each of the impurities over time is lower in GLN-1, as well as the percentage of total impurities.

The chemical name of the above-mentioned identified impurities is as follows:

| Impurity name | Chemical name |
|---|---|
| Impurity D | Des Gly³⁷ Liraglutide |
| Impurity E | D His⁷ Liraglutide |
| Impurity F | Des His⁷ Liraglutide |
| Impurity G | Des His⁷ - Ala⁸ Liraglutide |
| Impurity I | Des His⁷- Gly¹⁰ |
| Impurity H | Des His⁷- Glu⁹ |

### Example 3. Thioflavin T (ThT) Fibrillation Estimation Assay

Low physical stability of a peptide may lead to amyloid fibril formation, which is observed as well-ordered, thread-like macromolecular structures in the sample eventually resulting in gel formation. This has traditionally been measured by visual inspection of the sample. However, that kind of measurement is very subjective and depends on the observer. Therefore, the application of a small molecule indicator test is much more advantageous. Thioflavin T (ThT) is such a test and has a distinct fluorescence signature when binding to fibrils.

To minimise costs related to the active ingredient and being able to estimate the fibrils in the compositions (at 6 mg/mL) by ThT fibrillation assay, Bovine serum albumin (BSA) was used for the standard curve of the assay, since BSA has similar tendency to form fibrils, like liraglutide. To this aim, a BSA standard curve was prepared from 0.003 to 0.188 mM and a Thioflavin-T working solution at 50 μM. After preparation of standard concentration and one blank (PBS), the BSA standard samples at 80° C. for 30 min and then cool at room temperature for fibril generation.

### Preparation of the Samples

Preparation of standard Blank: Add 50 μL of heated and cooled PBS into 96 well plate in duplicate.
Preparation of Standard: Add 50 μL of heated and cooled BSA (0.003 to 0.188 mM) each into 96 well plate in duplicate.
Preparation of Liraglutide RLD/Test samples Blank: Add 50 μL of Milli-Q water into 96 well plate in duplicate.
Preparation of Liraglutide RLD/Test samples: Add 50 μL RLD/Test samples (50 μM) into 96 well plate in duplicate. Then read the plate as per method created in Micro plate reader.

After addition of all blanks, standard curve concentrations and liraglutide (RLD)/test sample concentrations in the plate, further 150 μL of thioflavin-T working solution (50 μM) were added into each well and mixed gently by pipetting. Read the plate as per the following microplate reader conditions:

(Read Mode: Fluorescence; excitation wavelength: 440 nm; emission wavelength: 482 nm; bandwidth excitation: 9 nm; bandwidth emission: 15 nm; plate type: 96 well standard opaque; plate height: 14.6 mm; plate Shake: 60 seconds, orbital, medium (before first read); PMT and optics: 6 flashes/read; Read from: Top; Read height: 1.00 mm).

The measurements were performed using a CLARIOstar® Plus fluorescence microplate reader.

### Calculations and Results

Plotted the standard curve with the blank subtracted values of standards on Y-axis and their concentrations on X-axis.

Derived slope (Mstd), intercept (Cstd) and regression (R2) from the straight-line equation.

The following compositions were measured according to the above ThT fibrillation assay:

| Composition | Temperature (° C.) | pH |
|---|---|---|
| GLN-1 | 8 | 8.15 |
| Comp. Ex. 2 | 60 | 7 |
| Comp. Ex. 3 | 80 | 7 |
| Comp. Ex. 4 | 60 | 8.15 |

GLN-1 was prepared as described in Example 1. Comp. Ex. 2-4 were prepared as Comp. Ex. 1, but the pH and/or temperature were modified according to the above table. After preparation, the samples were stored for 1 month at 30° C./Relative Humidity 65% and analysed. Afterwards, samples were analysed according to the ThT fibrillation assay detailed above and the following results were obtained:

| Composition | Fibrils content (μM) |
|---|---|
| GLN-1 | 0.0178 |
| Comp. Ex. 2 | 0.0392 |
| Comp. Ex. 3 | 0.0518 |
| Comp. Ex. 4 | 0.1217 |

### CITED INFORMATION

European Pharmacopoeia 10th edition, 2019.
ICH HARMONIZED TRIPARTITE GUIDELINE: STABILITY TESTING OF NEW DRUG SUBSTANCES AND PRODUCTS, Feb. 6, 2003 (“ICH Q1A (R2)”)
U.S. Pat. No. 8,748,376 B2, STABLE FORMULATIONS OF PEPTIDES, issued on Jun. 10, 2014, to Ludvigsen et al. of Novo Nordisk A/S (related to WO 2006/051110 A2).
WO 2006/051110 A2, STABLE FORMULATIONS OF PEPTIDES, published on May 18, 2006, to to Ludvigsen et al. of Novo Nordisk A/S (related to U.S. Pat. No. 8,748,376B2).
WO 2020/127476 A1, PHARMACEUTICAL COMPOSITION COMPRISING GLP-1 ANALOGUE, published on Jun. 25, 2020, to Roszman et al. of KRKA, D.D., Novo Mesto.
WO2021/123228 A1, PHARMACEUTICAL COMPOSITION COMPRISING GLP-1 ANALOGUE, published on Jun. 24, 2021, to Rozman et al. of KRKA, D.D., Novo Mesto.

The cited information is incorporated by reference to the extent necessary to understand embodiments and/or aspects disclosed herein. The subject matter of European patent application EP24382587, filed on May 31, 2024, is incorporated by reference in its entirety except that if a conflict exists between the incorporated subject matter and the subject matter disclosed herein, then the subject matter herein controls.

1. A method for the preparation of a pharmaceutical composition comprising a GLP-1 agonist, a tonicity modifier, a buffering agent, and a preservative which comprises the following steps:
a. preparing a final solution comprising the tonicity modifier, the buffering agent, the preservative, the GLP-1 agonist and water for injection; and
b. adjusting the pH of the final solution between 6.5-8.5, with a pH adjuster;
wherein the final solution is prepared at a temperature between 2-15° C.

2. The method according to claim 1, wherein the pH of the final solution is between 7.5-8.5.

3. The method of claim 1, wherein the final solution temperature does not exceed 15° C.

4. The method of claim 1, wherein the final solution is prepared by mixing a first solution comprising the tonicity modifier, the buffering agent, the preservative, and water for injection; and a second solution comprising a GLP-1 agonist and water for injection, wherein the first and second solutions are prepared at a temperature between 2-15° C.

5. The method of claim 1, further comprising bubbling nitrogen gas, until the dissolved oxygen content is <2.0 ppm.

6. The method of claim 1, further comprising filtering the final solution through a 0.2 μm pore size filter.

7. The method of claim 1, wherein the GLP-1 agonist comprises liraglutide, semaglutide, or tirzepatide.

8. The method of claim 1, wherein the tonicity modifier comprises propylene glycol.

9. The method of claim 1, wherein the buffering agent comprises sodium dihydrogen phosphate, dibasic sodium phosphate, sodium phosphate, phosphoric acid, acetic acid, sodium acetate, sodium bicarbonate, carbonic acid, sodium carbonate, citrate, citric acid, meglumine, glycine, histidine, lysine, arginine, asparagine, glutamic acid, sodium glutamate, tris (hydroxymethyl)-aminomethane, methionine, Hepes, maleic acid, malic acid, lactate, or mixtures thereof.

10. The method of claim 1, wherein the comprises phenol, m-cresol, methyl p-hydroxybenzoate, propyl p-hydroxybenzoate, benzoic acid, benzyl alcohol, benzyl benzoate, 2-phenoxyethanol, butyl p-hydroxybenzoate, 2-phenylethanol, benzyl alcohol, chlorobutanol, acetone sodium bisulfite, benzalkonium chloride, benzethonium chloride and thiomerosal, or any combinations thereof.

11. The method of claim 1, further comprising filling aseptically the final solution into cartridges flushed with nitrogen gas.

12. A pharmaceutical composition obtained by the method of claim 1.

13. The pharmaceutical composition obtained by the method of claim 1 wherein the fibrils concentration in the pharmaceutical composition after 1-month storage at 30° C. and 65% relative humidity is less than 0.02 μM as measured by Thioflavin T (ThT) Fibrillation Estimation Assay.

14. The pharmaceutical composition obtained by the method of claim 1 wherein the oxygen content is <2.0 ppm.

15. The pharmaceutical composition obtained by the method of claim 1 wherein the GLP-1 agonist is liraglutide.

16. The pharmaceutical composition obtained by the method of claim 1 wherein the GLP-1 agonist is semaglutide.

17. The pharmaceutical composition obtained by the method of claim 1 wherein the GLP-1 agonist is tirzepatide.

18. The pharmaceutical composition obtained by the method of claim 1 wherein said composition is filled into a cartridge.

19. A cartridge comprising the pharmaceutical composition obtained by the method of claim 1.

20. The cartridge comprising the pharmaceutical composition obtained by the method of claim 1, wherein no air bubbles remain inside the cartridge.
